(19) United States
(12) Patent Application Publication
(10) Pub. No.: US 2017/0348464 A1
(43) Pub. Date: Dec. 7, 2017

(54) COMPONENTS FOR FUSING VERTEBRAL BODIES

(71) Applicant: CeramTec GmbH, Plochingen (DE)

(72) Inventors: Heinrich WECKER, Eckental (DE); Alfons KELNBERGER, Röthenbach (DE); Mateusz JUSZCZYK, Velden (DE)

(73) Assignee: CeramTec GmbH, Plochingen (DE)

(21) Appl. No.: 15/327,906

(22) PCT Filed: Jul. 22, 2015
(86) PCT No.: PCT/EP2015/066776

§ 371 Date: Jan. 20, 2017

(30) Foreign Application Priority Data
Jul. 22, 2014 (DE) 10 2014 214 286

(51) Int. Cl. A61L 27/56; A61L 27/50; A61L 27/54; A61L 27/10; A61F 2/44; A61F 2/30

(52) U.S. Cl. CPC A61L 27/56; A61L 27/10; A61F 2/4455; A61L 27/50; A61L 27/54; A61F 2240/005; A61F 2/3094; A61L 2430/38

(57) ABSTRACT

The invention relates to components for fusing vertebral bodies and to methods for the production and use thereof.

Subject of the present invention are components for fusing vertebral bodies, methods for their production and their use.

Endoprosthetic components for fusing vertebral bodies have long been known. They are matched in their geometry to the anatomy of the human vertebral body, they are located between two vertebral bodies and partially or completely replace the intervertebral disk.

Typically, in a first phase of remaining in the human body, they hold the vertebral bodies apart and hence in an anatomically correct position solely due to their mechanical characteristics. As a result they promote the fusing and later the growth of the two surrounding vertebral bodies in a second phase.

Correspondingly known components for fusing vertebral bodies are based on metallic materials, such as tantalum or titanium for example.

Disadvantages of these metallic materials are, for example:
High risk of infection
Metallic abrasion and negative effects on the human organism resulting therefrom
Artifacts in imaging in medical diagnostics
Ageing effects and long-term behavior

Components based on plastics, such as highly cross-linked PE materials (polyethylene) or PEEK (polyetheretherketone), are also known.

Disadvantages of plastics are, for example:
Inadequate mechanical characteristics, such as the breaking-off of serrations, for example, or other characteristics of the component, e.g. when fitting. This can have negative effects on the human organism.
Lack of portrayability with common imaging processes (MRI, X-ray). As a result, this requires the use of metal markers.
Ageing effects and long-term behavior

Ceramic components, for example based on oxidic or non-oxidic ceramics, for example made of silicon nitride or aluminum-oxide-based materials, are also known.

A decisive further disadvantage of ceramic components for fusing vertebral bodies is, in general, the high rigidity, which is substantially higher than that of human vertebral bodies. As a result of the correspondingly low mechanical compliance of the components due to the high modulus of elasticity of the ceramic materials of the order of magnitude of a few 100 GPa, this can lead to an absence of the mechanical stimulus in the spinal column necessary for bone formation and bone remodeling, and the fusion of the vertebral bodies is only inadequate or fails to materialize in the long term. This phenomenon is also known under the term “stress shielding”. It can lead to the depletion of bone material and prevent the buildup of new bone material.

On the other hand, ceramic structures which are already porous and which can be produced by means of various methods, for example by means of template techniques/forming techniques, direct foaming methods or freeze-foaming methods, are also known. Ceramic components for fusing vertebral bodies can be produced with the above methods, and the bone cells have optimum structures for synthesizing new bone material, which, if necessary, can also be formed osteoinductively by additional functionalizing layers.

By this means, it should be possible to produce interconnecting, porous ceramics with adequate strength of an order of magnitude >10 MPa and, due to the structural buildup, mechanically compliant structures with elastic characteristics, which, with the physiologically common biomechanical loads, permit a change in length which is favorable for bone building and remodeling without at the same time damaging the ceramic structure.

It is also known that for bone remodeling—that is to say for bone preservation—changes in length of an order of magnitude of 0.1% are necessary and the re-formation of bones with deformations in a range of approximately 0.15% to 0.4% is required. Greater deformations of the bone lead to microfractures and finally to bone breakage.

It has also been shown that porous ceramic structures can be designed such that these mechanical conditions, which promote bone growth, can be produced.

However, a major disadvantage is that, with these porous structures based on ceramic materials, point loading can occur on the individual ligaments of the porous ceramic implant due to biomechanical loads when handling during implantation—for example due to the use of an instrument or during healing in the human body—and said implant can then fail at least locally.

In the worst case, the ceramic component can break. This therefore presents a dilemma.

Either the ceramic component is made sufficiently large to withstand the mechanical requirements as an implant—this can then suppress bone formation and it is only suitable to a limited extent as a ceramic fusion implant—or it is designed to be porous and bone-friendly; it may then be that it is not suitable for the mechanical requirements.

The object of the present invention is to guarantee sufficiently large elongation to promote bone re-formation and bone renewal and at the same time high mechanical and adequate stability in a ceramic component for fusing vertebral bodies.

The invention relates to a ceramic component for fusing vertebral bodies in the area of the human spinal column (cages) which, on the one hand, supports bone re-formation and bone remodeling solely due to its mechanical characteristics and, on the other, has adequately high mechanical stability and a high level of protection against forces and effects which occur during implantation and while it remains in the human body.

Furthermore, the ceramic implant is to ensure rapid and sustainable fusion of two vertebral bodies and thus provide an optimum result for patients.

The object according to the invention is achieved by the following characteristics of the invention: Component for fusing vertebral bodies, wherein the component is made from a porous, multi-surface body (second region) which has an edge with a sealed region (first region) on at least one surface. Preferably, aluminum oxide, zirconium oxide or mixed ceramics based on the previously mentioned materials are used as the material for the ceramic of the first region.

Within the framework of this invention, mixed ceramics include zirconium-oxide-reinforced materials (ZTA, zirconia-toughened alumina) among others. Zirconium oxides include all kinds of tetragonally stabilized zirconium oxides, such as for example yttrium-, cerium- or gadolinium-stabilized zirconium oxides.

In particular, zirconium-oxide-based materials with a modulus of elasticity of >100 GPa and therefore the order of magnitude of steel are suitable for the invention. As these materials have high strengths, it is ensured that the component as a whole has adequate and inventively high strength. The 4-point bending strengths of these materials measured as standard lie in the range between 500 and 2000 MPa, preferably 700 to 1500 MPa.

According to the invention, the second inner region is made from a porous ceramic, wherein, in principle, the ceramic can comprise the same material as the first region but be bioactivated by the supplementary addition of bioactive substances.

According to the invention, by way of example, layers or admixtures of known bioactive substances based on calcium phosphate, such as hydroxylapatite or tricalcium phosphate (TCP) for example, and also glass-like substances, such as bioglasses for example, can be considered for this purpose. With regard to the hydroxylapatite variants, according to the invention, nano-particle-based coatings in particular have been shown to be particularly favorable.

The best-known, and for these purposes best-suited, bioglass has the designation 45S5 with the main constituents SiO2 (silicon dioxide), CaO (calcium oxide), Na2O (sodium superoxide) and P2O5 (phosphorus pentoxide). However, there are a wealth of bioglass compounds which, for example, also have antibacterial properties, which, in turn, drastically reduces the risk of infection.

According to the invention, phosphating layers, which are bonded covalently to the ceramic surface and are extremely attractive for bone cells due to their high hydrophilicity, represent a further method of bioactivation.

According to the invention, this bioactivation ensures that the process of bone re-formation is efficiently stimulated and leads to a permanent fusion of the two vertebral bodies.

The inner structure of this second inner region has a decisive influence on the osteoconductivity of the component and consequently on the ability to synthesize new bone material. So-called trabecular structures have been shown to be particularly suitable. These structures are very similar to the morphology of spongiform bones and ensure optimum conditions for growth of the cells involved with bone synthesis.

Here, the following parameters are decisive according to the invention:
Pore distributions with a maximum between 100 and 1000 μm, preferably between 400 and 600 μm
Porosities between 75 and 85% v/v
Interconnectivity, i.e. the individual pores are connected to one another and enable optimum vascularization of the component

These trabecular structures can be produced by means of known forming technologies, wherein polyurethane carrier structures, which are immersed in appropriately prepared ceramic slurry and subsequently burnt out, are used as a template.

Direct foaming methods, with which the foam-forming substances are part of the ceramic slurry and the porous interconnecting structures can be specifically produced spontaneously, are likewise conceivable.

The mechanical characteristics of the second inner region in the finished sintered state depend greatly on the material composition and on the structure, but can therefore also be specifically adjusted and matched to the biological environment. According to the invention however, the second region of the component is less strong and less rigid than the first, outer region of the component to enable the deformations necessary for stimulation of the bone to take place. In particular zirconium-oxide-based materials, such as for example cerium-stabilized zirconium oxide, and also multi-phase zirconium-based materials, such as for example yttrium-stabilized zirconium oxide containing strontium-hexaaluminate, are suitable for the invention due to their relatively low modulus of elasticity. Here, the modulus of elasticity of the porous inner region (second region) lies in the range of <100 GPa, preferably <50 GPa, particularly preferably <25 GPa, quite preferably particularly between 1 and 10 GPa. Here, the effective, macroscopic modulus of elasticity of the porous structure is understood to be the modulus of elasticity of the inner region.

The corresponding compressive strengths lie in the region of a few 10 MPa. Here, the compressive strength of the material of the sealed region (first region) is at least a factor 10 higher than the compressive strength of the porous region (second region).

The combination of these two regions, or the design of the component resulting from the combination, is decisive for the component according to the invention.

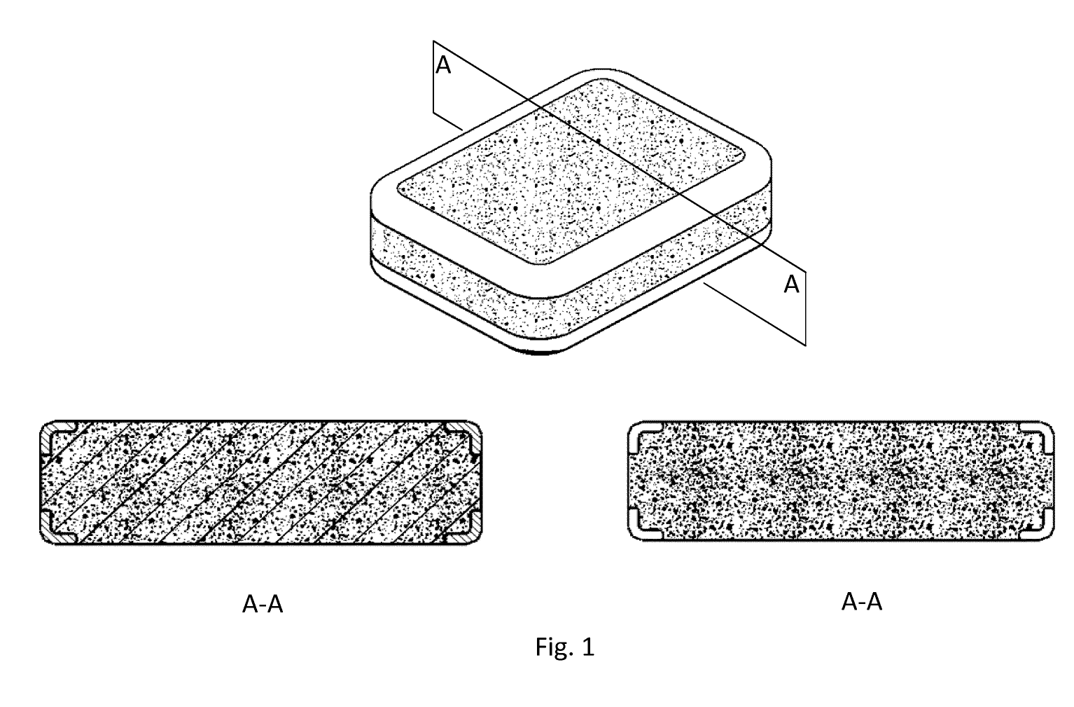

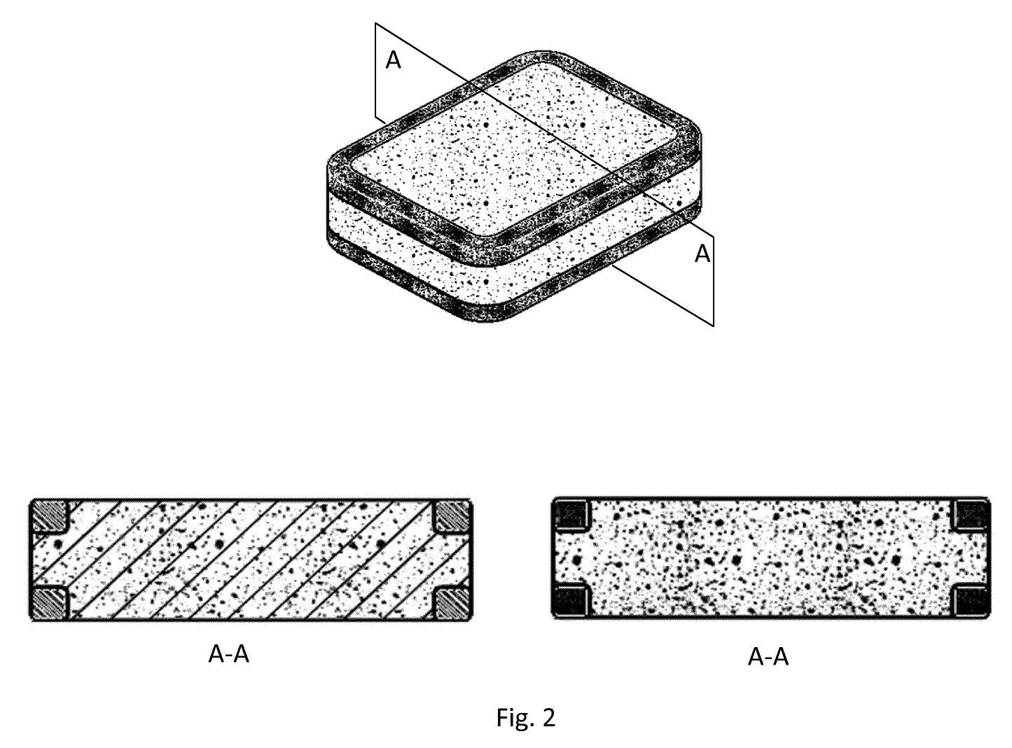

In a first variant according to the invention, the first region is formed in two parts, wherein, in each case, one part of the first region circumferentially encompasses the mechanically sensitive edge regions of the face surfaces of the second region according to FIGS. 1 and 2.

The two circumferential parts of the first region are securely connected to the porous second region, for example sintered together. However, the two parts of the first region have no connection to one another.

According to the invention, the mechanical compliance of the component is therefore provided by the porous second region, wherein the mechanically sensitive edge regions of monolithic, solid and circumferential second ceramic are protected.

In addition, in this variant according to the invention, the second porous region as a whole is offset inwards and is thus protected from the two parts of the first region in the manner of a protector.

According to the invention, the two parts of the first region can also have structures, for example cutouts, holes with/without thread or grooves, which enable the engagement of an instrument for safe handling, in particular during implantation.

According to the invention, the interface with the instrument therefore extends primarily over the stable first region.

In the same way, the first region of the component according to the invention constitutes the primary interface with the two end plates of the adjacent vertebral bodies, which, by means of suitable structures such as serrations, edges or pyramid-shaped elevations for example, ensure optimum primary stability.

According to the invention, the connection of the two regions can be made by joining in the green state and subsequent co-sintering, thus resulting in a substance-to-substance bond.

In principle, according to the invention, any kind of shape in the green according to the prior art is suitable for ensuring a connection of the two regions. In particular, the porous second region can be foamed directly in the two monolithic sub-regions, which are arranged in a special mold.

Naturally, instead of being made from ceramic, such an implant can also be produced from other materials which are suitable for implantation purposes, for example metals and metal wires made from titanium. However, several of the advantages listed above then no longer apply, such as fewer artifacts in the imaging, the general non-toxicity and others. The same also applies to the next variant.

In a second variant according to the invention according to FIGS. 3 and 4, the sensitive edge regions of the porous ceramic of the second region are infiltrated with a ceramic slurry in the green state. As a result of the infiltration, these regions are sealed. A substance-to-substance bond, with which the mechanically sensitive regions of the fusion component are protected, is formed in the subsequent sintering.

According to the invention, in particular rapid prototyping methods according to the prior art are suitable for this variant, as these enable the implant to be built up step-by-step and the two regions according to the invention can be formed independently of one another.

In particular, according to the invention, specific gradients in the porosity or other porous structures can also be set up or produced with this method in order to specifically match the elastic characteristics of the ceramic component to the mechanical requirements of the bone growth.

In particular, the invention includes a component for fusing vertebral bodies, wherein the component is made from a porous, multi-surface body which has an edge with sealed region on at least one surface.

In a preferred embodiment, the component consisting of the porous, multi-surface body in each case has a sealed region on at least one surface on two opposing edges.

In a further preferred embodiment, the component consisting of the porous, multi-surface body has an edge with a circumferential sealed region on at least one surface.

In a further preferred embodiment, the component consisting of the porous, multi-surface body has edges with sealed regions on two mutually opposing surfaces.

In a further preferred embodiment, the component consisting of the porous, multi-surface body in each case has edges with a circumferential sealed region on two mutually opposing surfaces.

In a further preferred embodiment, the component consisting of the porous, multi-surface body is made of porous ceramic.

In a further preferred embodiment, the component consisting of the porous, multi-surface body is made of porous ceramic and permits large elastic elongations.

In a further preferred embodiment of the component consisting of the porous, multi-surface body, the sealed region is made of densely sintered ceramic.

In a further preferred embodiment of the component consisting of the porous, multi-surface body, the sealed region is made of densely sintered ceramic with high mechanical stability.

In a further preferred embodiment of the component consisting of the porous, multi-surface body, the material for the ceramic is chosen from the group consisting of aluminum oxide, zirconium oxide or mixed ceramics based on those mentioned above.

In a further preferred embodiment of the component consisting of the porous, multi-surface body, the material for the ceramic is chosen from zirconium-oxide-reinforced materials (ZTA, zirconia-toughened alumina).

In a further preferred embodiment of the component consisting of the porous, multi-surface body, the workpiece for the ceramic is tetragonally stabilized zirconium oxide.

In a further preferred embodiment of the component consisting of the porous, multi-surface body, the workpiece for the ceramic is yttrium-, cerium- or gadolinium-stabilized zirconium oxide.

In a further preferred embodiment of the component consisting of the porous, multi-surface body, the material for the porous ceramic is chosen from the group consisting of aluminum oxide, zirconium oxide or mixed ceramics based on those mentioned above, wherein the material additionally contains a bioactive substance by means of which the ceramic is bioactivated.

In a further preferred embodiment of the porous, multi-surface body, the bioactivation is carried out by a bioactive substance based on calcium phosphate, by a glass-like substance or by a phosphating layer.

In a further preferred embodiment of the component consisting of the porous, multi-surface body, the bioactive substance is based on calcium phosphate, hydroxylapatite or tricalcium phosphate (TCP).

In a further preferred embodiment of the component consisting of the porous, multi-surface body, the glass-like substance is a bioglass with the designation 45S5, which contains SiO2 (silicon dioxide), CaO (calcium oxide), Na2O (sodium superoxide) and P2O5 (phosphorus pentoxide).

In a further preferred embodiment of the component consisting of the porous, multi-surface body, the effective macroscopic modulus of elasticity of the porous inner region (second region) is <100 GPa.

In a further preferred embodiment of the component consisting of the porous, multi-surface body, the sealed region has a 4-point bending strength in the range from approximately 500 to 2000 MPa, preferably 700 to 1500 MPa.

In a further preferred embodiment of the component consisting of the porous, multi-surface body, the material for the porous ceramic consists of a zirconium-oxide-based material with a compressive strength of >10 MPa.

In a further preferred embodiment of the component consisting of the porous, multi-surface body, the material of the sealed region has a compressive strength which is at least a factor 10 higher than the porous region.

In a further preferred embodiment of the component consisting of the porous, multi-surface body, the porous ceramic has the following parameters:
a pore size between 100 and 1000 μm,
a porosity between 75 and 85% v/v, and
an interconnectivity of the pores.

In a further preferred embodiment, the geometry of the component is matched to the anatomy of the human vertebral body.

The method for producing the component according to the invention consisting of the porous, multi-surface body includes the use of template techniques/forming techniques, direct foaming methods or freeze-foaming methods.

In a preferred embodiment of the method, the edge regions (edges) of the porous ceramic are sealed in a second step by infiltration with a ceramic slurry.

1. A component for fusing vertebral bodies, wherein the component is made from a porous, multi-surface body which has an edge with sealed region on at least one surface.

2. The component according to claim 1, wherein the component is made from a porous, multi-surface body which in each case has a sealed region on at least one surface on two opposing edges.

3. The component according one of the preceding claims, to claim 1, wherein the component is made from a porous, multi-surface body which has an edge with a circumferential sealed region on at least one surface.

4. The component according to claim 1, wherein the component is made from a porous, multi-surface body which has edges with sealed regions on two mutually opposing surfaces.

5. The component according to claim 1, wherein the component is made from a porous, multi-surface body which in each case has edges with a circumferential sealed region on two mutually opposing surfaces.

6. The component according to claim 1, wherein the porous, multi-surface body is made from porous ceramic.

7. The component according to claim 1, wherein the porous, multi-surface body is made from porous ceramic and permits large elastic elongations.

8. The component according to claim 1, wherein the sealed region is made from densely sintered ceramic.

9. The component according to claim 1, wherein the sealed region is made from densely sintered ceramic with high mechanical stability.

10. The component according to claim 1, wherein the material for the ceramic is chosen from the group consisting of aluminum oxide, zirconium oxide or mixed ceramics based on those mentioned above.

11. The component according to claim 1, wherein the material for the ceramic is chosen from zirconium-oxide-reinforced materials (ZTA, zirconia-toughened alumina).

12. The component according to claim 1, wherein the material for the ceramic is tetragonally stabilized zirconium oxide.

13. The component according to claim 14, wherein the material for the ceramic is yttrium-, cerium- or gadolinium-stabilized zirconium oxide.

14. The component according to claim 1, wherein the material for the porous ceramic is chosen from the group consisting of aluminum oxide, zirconium oxide or mixed ceramics based on those mentioned above, wherein the material additionally contains a bioactive substance by means of which the ceramic is bioactivated.

15. The component according to claim 1, wherein the bioactivation is carried out by a bioactive substance based on calcium phosphate, by a glass-like substance or by a phosphating layer.

16. The component according to claim 17, wherein the bioactive substance is based on calcium phosphate, hydroxylapatite or tricalcium phosphate (TCP).

17. The component according to claim 17, wherein the glass-like substance is a bioglass with the designation 45S5, which contains SiO₂ (silicon dioxide), CaO (calcium oxide), Na₂O (sodium superoxide) and P₂O₅ (phosphorus pentoxide).

18. The component according to claim 1, wherein the effective macroscopic modulus of elasticity of the porous inner region (second region) is <100 GPa.

19. The component according to claim 1, wherein the sealed region has a 4-point bending strength in the range from approximately 500 to 2000 MPa, preferably 700 to 1500 MPa.

20. The component according to claim 1, wherein the material for the porous ceramic consists of a zirconium-oxide-based material with a compressive strength of >10 MPa.

21. The component according to claim 1, wherein the material of the sealed region has a compressive strength which is at least a factor 10 higher than the porous region.

22. The component according to claim 1, wherein the porous ceramic has the following parameters:
a pore size between 100 and 1000 μm,
a porosity between 75 and 85% v/v, and an interconnectivity of the pores.

23. The component according to one of the preceding claims, characterized in that the geometry of the component is matched to the anatomy of the human vertebral body.

24. A method for producing the component according to claim 1, wherein production is carried out using template techniques/forming techniques, direct foaming methods or freeze-foaming methods.

25. The method for producing the component according to claim 24, wherein the edge regions (edges) of the porous ceramic are sealed in a second step by infiltration with a ceramic slurry.
